(19) United States
(12) Patent Application Publication
(10) Pub. No.: US 2021/0217531 A1
(43) Pub. Date: Jul. 15, 2021

(54) CLOUD-CONNECTED RING-BASED SENSOR SYSTEM

(71) Applicant: Yogendra Kumar BOBRA, San Jose, CA (US)

(72) Inventor: Yogendra Kumar BOBRA, San Jose, CA (US)

(21) Appl. No.: 17/134,423

(22) Filed: Dec. 27, 2020

Related U.S. Application Data
(60) Provisional application No. 62/960,776, filed on Jan. 14, 2020.

(51) Int. Cl. G16H 50/30; G16H 50/20; G16H 10/60; G06F 1/16

(52) U.S. Cl. CPC G16H 50/30; G06F 1/163; G16H 10/60; G16H 50/20

(57) ABSTRACT

A cloud-connected ring-based sensor system includes a ring, a ring-manager application (e.g., running on a mobile device), and cloud services. The ring collects sensor data and forwards it to the app, which forwards it (modified or unmodified) to the cloud services. The cloud services can analyze the data, e.g., to look for trends. The cloud services can provide access to the data and analysis results to a provider, who can then return information (instructions, advice, etc.) to the cloud services, which makes the information available to the ring-wearer via the ring-manager application. The sensor can be a heartbeat sensor. In that case, the ring automatically switches between an active mode and a standby mode depending on whether or not a heartbeat is detected.

### BACKGROUND

Heart disease is the leading cause of death in the United States and throughout the world. Early detection, accurate diagnosis, and carefully monitored treatment are key to limiting the seriousness of heart disease. However, these practices can be problematic to implement. For example, arrhythmias (heartbeat irregularities) can be indicative of and useful in diagnosing heart disease and stroke. However, some arrhythmias are not detected by the person afflicted; also, arrhythmias are usually intermittent and, so, may escape detection by a doctor during a visit. Portable heart monitors and heart-monitoring patches are available, but impose a level of inconvenience that limits their usefulness, because these can't be used extended period of time, weeks and months. What is needed is a convenient and effective way to monitor heart health, over very long periods of time under normal daily living conditions.

### BRIEF DESCRIPTION OF THE DRAWINGS

FIG. 1 is a schematic diagram of a cloud-connected ring-based sensor system.

FIG. 2 is a flow chart of a cloud-connected ring-based sensor process implementable in the system of FIG. 1 and in other systems.

FIG. 3 is a gray-scale photograph of a ring incorporable in the system of FIG. 1.

FIG. 4 is a gray-scale photograph of the ring of FIG. 3 seated in a charging station.

FIG. 5A is a schematic diagram of the ring of FIG. 3; FIGURE SB is a flow chart of a power-manager process implementable in the ring of FIG. 3.

FIG. 6 is a schematic diagram of a ring-manager application for the ring of FIG. 3.

FIG. 7 is a gray-scale screen shot of a display by the ring manager application of FIG. 6.

FIG. 8 is a schematic diagram of cloud services for the ring of FIG. 3.

FIG. 9 is a schematic diagram of an expert system of the cloud services of FIG. 8.

### DETAILED DESCRIPTION

The present invention provides a cloud-connected ring-based sensor system including a ring, a ring-manager application, and cloud services. The ring, when worn on the finger of a human “ring-wearer”, senses a phenomenon, collects and processes sensor data, and transmits processed sensor data to the ring-manager application (e.g., running on a smartphone or other mobile device), which forwards processed sensor data to the cloud services.

The cloud services provide sophisticated analysis of processed sensor data including identifying patterns and trends and putting the sensor data in historical and normative contexts. The cloud services can provide real-time cloud-based access to the sensor data and analysis results to providers, who in turn can input information for the ring-wearer to the cloud services, which forward the provider information to the ring-manager application for display to the ring-wearer.

The ring can detect whether or not it is mounted on a finger and transition to an active mode when it is donned and transition to a low-power mode when it is removed (to extend the time available between successive battery recharge needs). In active mode, the ring collects sensor data on a continuing basis. In low-power mode, the ring does not collect data except to check, e.g., periodically, whether or not it is mounted on a finger.

Each ring can include a sensor set of one or more sensors. Embodiments of the invention can employ a wide range of different sensors including sensors for monitoring health or other parameters relating to the wearer and sensors for monitoring the wearer's environment. In one of its aspects, the invention provides a ring that includes a sensor for tracking inter-beat intervals (IBI) is described in detail below; in that embodiment, the sensor used to track inter-beat intervals is also used to detect whether or not the ring is mounted on a finger so that the ring can be switched from a low-power mode to an active mode when it is donned.

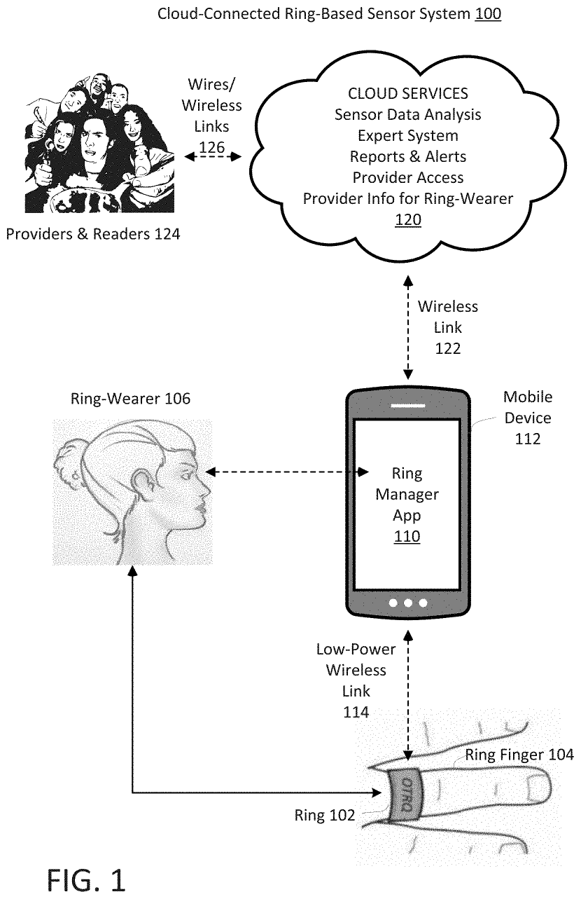

A cloud-connected ring-based sensor system 100 includes a ring 102, which is shown, in FIG. 1, mounted on a finger 104 of a ring-wearer 106. A ring-manager application (app) 110 running on a mobile device 112 manages ring 102, with which it communicates via a low-power (e.g., Bluetooth) link 114. Sensor data can be uploaded from ring 102 to app 110 via link 114. Mobile device 112 may be, for example, a smartphone owned by ring wearer 106 so that app 110 can audio-visually communicate sensor data and related statistics to ring wearer 106.

Ring manager app 110 can upload sensor data to cloud services 120 via a wireless link 122. Cloud services 120 can provide controlled access to sensor data to users including service providers 124 and readers (read-only users such as researchers) over wired and/or wireless links 126. Providers 124 can upload information (e.g., insights, instructions, recommendations) for ring wearer 106 to cloud services 120 via links 126. Cloud services 120 can also generate such information, e.g., using an expert system. Cloud services 120 can then download such information to ringer manager app 110 over link 122; ring-manager app 110 can then display the information to ring wearer 106. In addition, providers 124 and/or cloud services 120 can provide updates for ring-manager app 110 and ring 102. These updates can be transmitted via links 122 and 114.

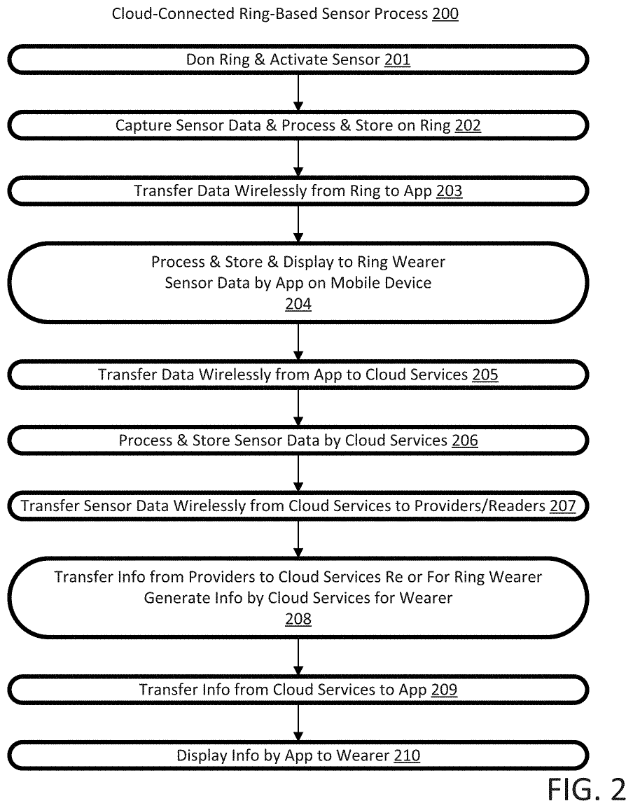

A cloud-connected ring-based sensor process 200, flow-charted in FIG. 2, can be implemented in system 100 and other systems. At 201, a ring-wearer dons a sensor ring. The ring, when off the finger, can be in a standby mode in which it is ready to detect when it is mounted on a finger, in which case, it is switched to an active mode that allows other events to be detected. Such automated activation avoids wearer errors such as forgetting to activate the ring, accidently de-activating the ring, and leaving the ring activated (and wasting battery power) when the ring is removed. In alternative embodiments, the ring includes an activation switch so that the ring wearer can activate and deactivate the ring.

In a heart-health embodiment, a ring includes a heartbeat sensor, a temperature sensor, and motion detector. The heartbeat sensor includes a red-light emitter, an infra-red light emitter, and a photodetector. The emitters periodically transmit pulses of light through the skin (e.g., at 100 pulses per second each), while the photodetector detects reflections of the emitted light. Reflections of red light are used to assess the flow of oxygen-rich blood, while reflections of the infra-red light are used to assess the flow of oxygen-depleted blood. The periodic pulses of infra-red light are out-of-phase with respect to the periodic pulses of red light so that the photodetections can be attributed according to their respective timings to the oxygenated blood or the oxygen depleted blood. Accordingly, the raw output of the heartbeat sensor includes two interleaved series of reflection magnitudes, with one series corresponding to the flow of oxygenated blood and the other corresponding to the flow of oxygen-depleted blood.

At 202, the activated ring captures raw sensor data, which is processed by the ring and stored on the ring. In some embodiments, the processed data is the same as the raw data; for example, the processing can simply involve organizing the raw data for storage. In other embodiments, the processing generates new data derived from the raw data so that the processed data differs from the raw data. In one variation, the raw data is retained along with the processed data; in another variation, the raw data is discarded once the processed data has been generated.

In the heart-health embodiment, the red and infra-red raw data streams are processed to detect and time-stamp heartbeats and blood oxygen level. In some variations, the processing can further involve subtracting time-stamps to determine inter-beat intervals (IBI), which in turn can be processed to determine heart-rate variability and to detect missing heartbeats. Variations differ in exactly what processing is performed on the ring and what processing is performed by the ring-manager app. In general, however, the raw data is not retained on the ring so that the more space efficient processed data can be collected and stored over a longer duration, e.g., over two or three days. In one heart-health embodiment, the IBI data is also discarded in favor of the parameter values derived from it.

At 203, the ring-processed sensor data is transferred wirelessly from the ring to the ring-manager app. While the ring-processed sensor data can be generated on a continuing basis, it need not be and is generally not uploaded to the app continuously. Rather the ring-processed sensor data can be uploaded in batches, e.g., an hour's or day's worth of data can be uploaded at a time to the app. This batch uploading allows for times when the mobile device on which the app runs is unavailable, out of power, or otherwise occupied.

At 204, the ring-processed sensor data can be processed and stored on by the ring-manager app; in addition, sensor data can be displayed by the app to the ring wearer. Depending on the embodiment, the resulting app-processed sensor data can be the same as or different from the ring-processed sensor data. In the heart-health example, if the ring evaluated all the parameters of interest, there might be nothing else for the app to determine. On the other hand, there might be parameters, e.g., heart-rate variability left to the app to calculate. The app can also include algorithms for detecting conditions that require prompt action by the ring-wearer, e.g., if the ring battery is running low or a provider should be contacted regarding abnormal readings.

In some variations, the ring-manager app stores data from previous transfers from the ring, in which case, the ring-manager app can provide statistical data covering longer time spans than is represented by the most-recent upload. For example, the ring-manager app could indicate how the most current heart-rate variability compares to previously determined heart-rate variabilities. In other variations, such inter-batch analyses are reserved for cloud services.

At 205, the app-processed sensor data is transferred wirelessly from the ring-manager app to cloud services. At 206, the app-processed sensor data is processed and stored by the cloud services. The cloud services can have much more storage capacity than either the app or the ring, so it can hold historical data that can be used to identify trends and perform other baseline comparisons. In some cases, the cloud-processed data can include the app-processed data, which it can supplement with trends and other data. In addition, the cloud services can have access to normative data to offer comparisons to data from the ring-wearer.

At 207, the cloud services provide controlled access to cloud-processed sensor data to users including providers and readers (e.g., researchers). Depending on the embodiment and the scenario, the transfers can be initiated by the cloud services or by the provider/reader. In the heart-health example, a medical professional or family member can be subscribed to continuously monitor a ring-wearing patient. The data transfer can be limited to alerts, batch summaries, or more detailed data. Otherwise, providers and other users can request access to cloud-processed data when needed.

At 208, providers can transfer to cloud services information (e.g., analysis results, instructions, recommendations) intended for the ring wearer. Cloud services can process this data to yield cloud-processed provider information. The cloud processed provider information can be the same as the provider information or differ in some way. For example, the cloud services may add metadata (time-stamp, identity of provider) to the provider data. Also, cloud services can generate information for the ring-wearer independent of provider input, e.g., using its expert system.

At 209, the cloud-processed provider information (and/or cloud processed information) is transferred from cloud services to the ring-manager app, which displays the information to the ring wearer at 210. In addition, cloud services can transfer updates to the ring manager app and/or to the ring firmware. For example, algorithms used to process sensor data can be updated, e.g., based on results from machine learning implemented by cloud services.

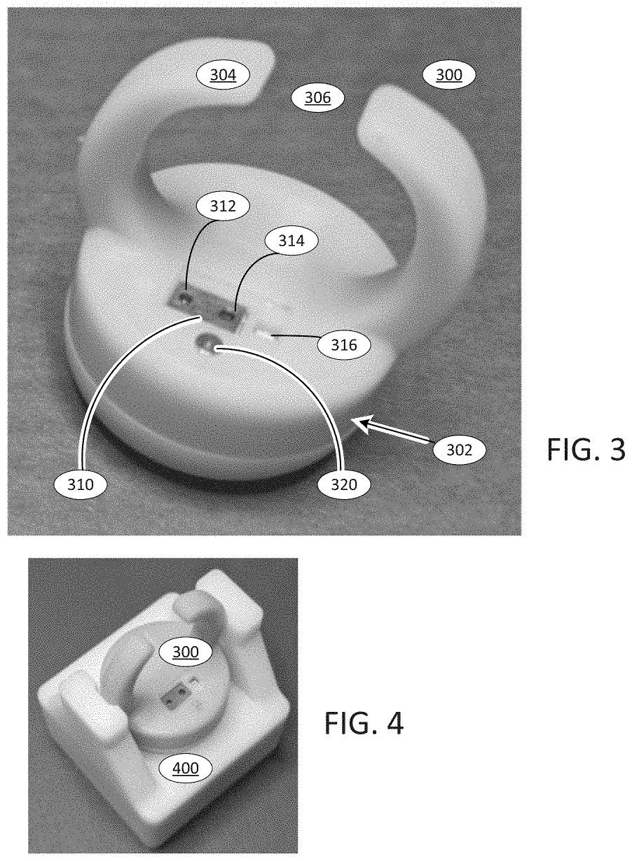

A sensor ring 300, shown in FIG. 3, tracks heart-health parameters including continuous heart rate, inter-beat interval (IBI), missing heart beats, and blood oxygen saturation. Ring 300 includes a head 302 and a shank 304. The shank 304 for ring 300 is split leaving a gap 306 so that arms of the shank can be urged apart to accommodate a range of finger perimeter lengths. In an alternative embodiment, the ring is custom fit and the shank is not split. In another alternative embodiment, the shank is not split, but is attached at the head at only one end to allow flexibility to accommodate different finger perimeters.

To track the heart-health parameters, ring 300 includes a heartbeat sensor 310 located on the back of ring head 302. Heartbeat sensor 310 includes red and infra-red light-emitting diodes (LEDs) 312 and 316, respectively, and a photo-detector 314. Light from LEDs 312 and 316 shines through the skin, and photo-detector 314 measures the amount of light that reflects back; the light reflections vary as blood pulses under the skin past the light. Each LED 312, 316 pulses at 100 pulses/second. The respective pulse streams for LEDs 312 and 316 are 180° out-of-phase so their reflections can be differentiated by time of arrival. The heartbeat detections are used to track continuous heart rate, inter-beat interval (IBI), and missing heart beats. In addition, heartbeat sensor 310 can be used to measure blood oxygen saturation. An excessive inter-beat interval indicates a missing heartbeat. Absence of a heart-beat detection for 3-5 seconds can indicate that the ring is not mounted on a finger—in which case, a switch to low-power mode can be triggered.

Also, on the back of head 302 is a gold contact 320 that makes contact with skin when ring 300 is donned on a finger-wearer's finger. This contact conducts heat so that skin temperature can be measured. Another temperature sensor, located within ring 300, is used to detect ambient temperature. Body temperature is calculated based on the skin temperature and the ambient temperature. An internal motion sensor is used to track physical activities and provide fall detection.

As shown in FIG. 4, a charger 400 is provided to charge an internal rechargeable battery in ring 300. This charging occurs via a couple of gold-plated contacts on the front face of ring 300. The ring battery lasts two-to-three days of continuous use; the charger can charge the battery in about two hours. To provide more continuous monitoring, a second ring can be alternated with ring 300.

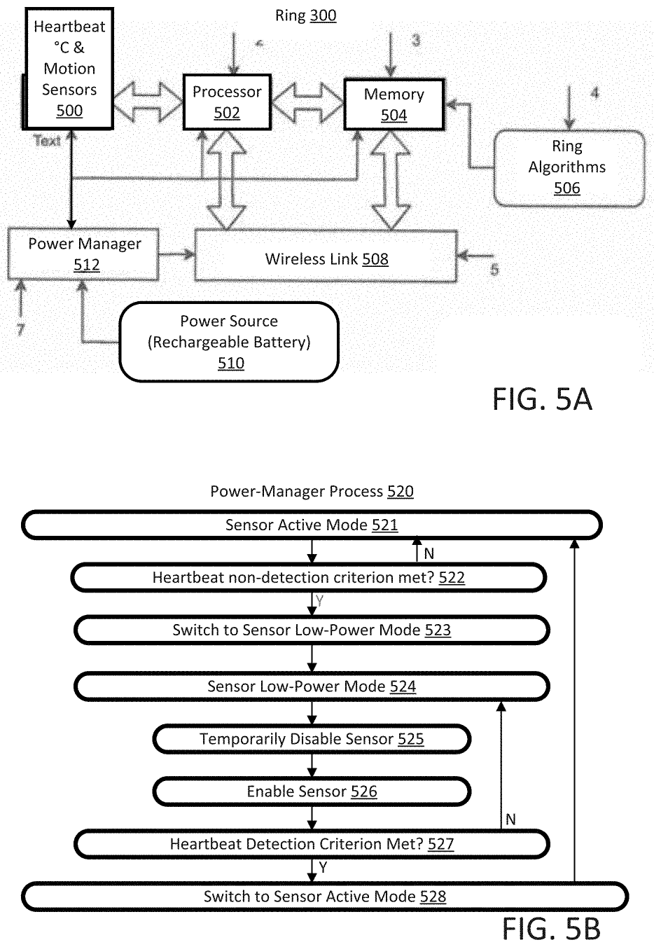

As shown in FIG. 5A, ring 300 includes heartbeat, temperature, and motion sensors 500, processor 502 memory 504, algorithms 506, a wireless link 508, a power source (e.g., rechargeable battery) 510, and a power manager 512. Sensors 500 include heartbeat sensor 310, and the body and ambient temperature sensors.

Processor 502 applies algorithms 506, which are stored in memory 504, to the raw sensor data to obtain inter-beat intervals and blood-oxygen levels. The inter-beat intervals are then used to calculate heart rates and heart-rate variability and to detect missing heartbeats. The raw data streams and the inter-beat intervals can be discarded to save memory capacity, while the blood oxygen level, heart-rate, heart-rate variability, and number of missing heart beats are stored in memory along with body and ambient temperatures, step counts and fall detections. Processor 502 transfers the processed sensor data to the ring-manager app via wireless link 508, provided the wireless link can detect the ring-manager app

Power manager 512 implements power-manager process 520, flow-charted in FIG. 5B. At 521, the ring is in sensor active mode 521 in which blood flow is monitored at a rate of 40-400 samples per second. At 522, during sensor active mode 521 a determination is made whether or not a heartbeat non-detection criterion is met. The heartbeat non-detection criterion can be, for example, failure to detect a heartbeat for some period of time long enough to exclude the possibility that one or more heartbeats have been skipped. The duration can be, for example, a minute. If the heartbeat non-detection criterion is not met, sensor active mode 521 continues and the criterion check at 522 is repeated until the criterion is met, e.g., because the ring has been removed from the finger.

In the event that, at 522, the heartbeat non-detection criterion is met, then the power manager switches to sensor low-power mode at 523. In low-power mode at 524, the sensor is temporarily disabled at 525, e.g., to save battery power. After 10-15 seconds, the sensor is enabled at 526.

At 527, with the sensor enabled, a check is made to see if a heartbeat detection criterion is met. For example, the criterion can be met when a heartbeat detection is made within 5 seconds. In the event that no heartbeat is timely detected at 527, low-powered mode 524 is continued and the loop 524-527 is repeated. However, in the event that a heartbeat is timely detected at 527, power manager switches to sensor active mode at 528, returning process 520 to 521. Power-manager process 520 relieves the ring-wearer of the burden of ensuring that the ring sensors are active when the ring is mounted on a finger and of ensuring that the ring is in standby mode to save power when the ring is off the finger.

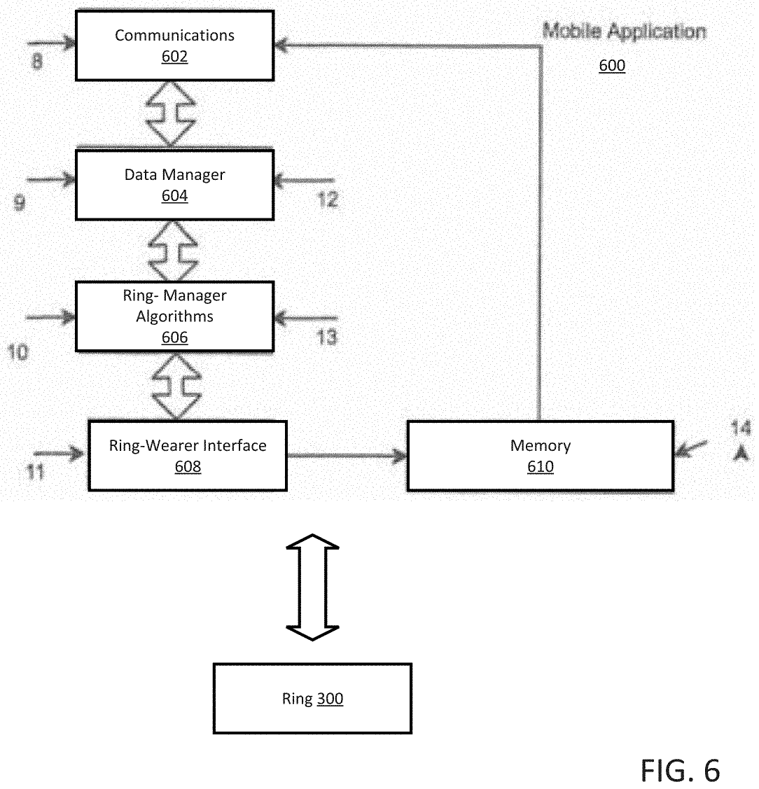

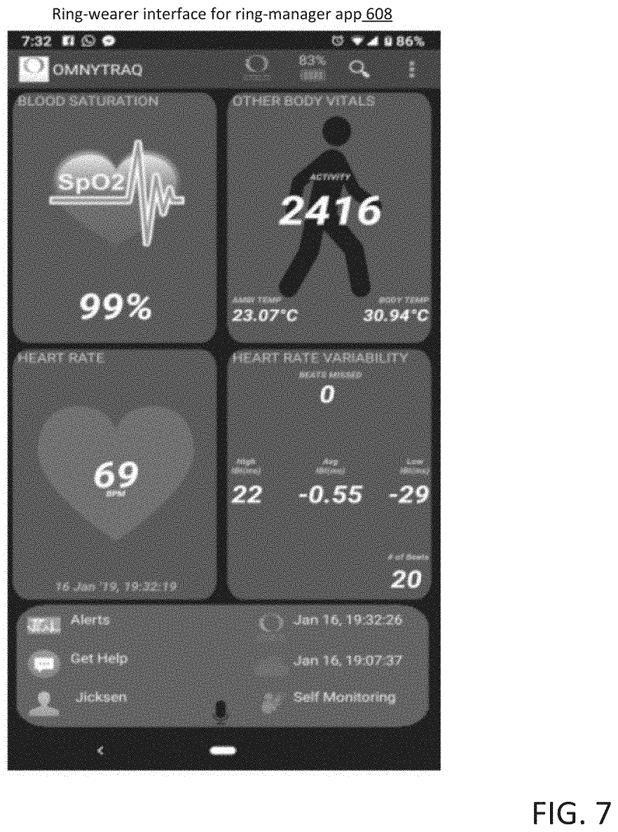

As shown in FIG. 6, a ring-manager app 600, which can run on a smartphone or other mobile device, includes a secure communications interface 602, a HIPAA (The Health Insurance Portability and Accountability Act of 1996) compliant data manager 604, ring-manager algorithms 606, and a user (e.g., ring-wearer) interface 608. Communications interface 602 provides for secure communication with ring 300 and cloud services. Ring 300 can detect when ring-manager app 600 is available and ready to receive sensor data; accordingly, ring 300 triggers sensor data uploads as appropriate. In addition, communications interface 602 can upload sensor data user-settings to cloud services.

Data manager 604 provides HIPAA-compliant access to data stored in memory 610, which data includes sensor data, other health data (via cloud services), and user profile data. Ring-manager algorithms 606 provide for calculating inter-beat intervals (IBI) based heart-rate variability, detecting missing heart beats, creating trend charts, and in some instances generating real-time alerts. Ring-wearer interface 608 provides for user input, e.g., by touch and voice, and audio-visual output.

An example screen shot from interface 608 is presented in FIG. 7. Interface 608 can display values for blood saturation, steps taken, ambient temperature, body temperature, heart rate, and heart rate variability values including high inter-beat interval, average inter-beat interval, low inter-beat interval, number of beats, and number of beats missed.

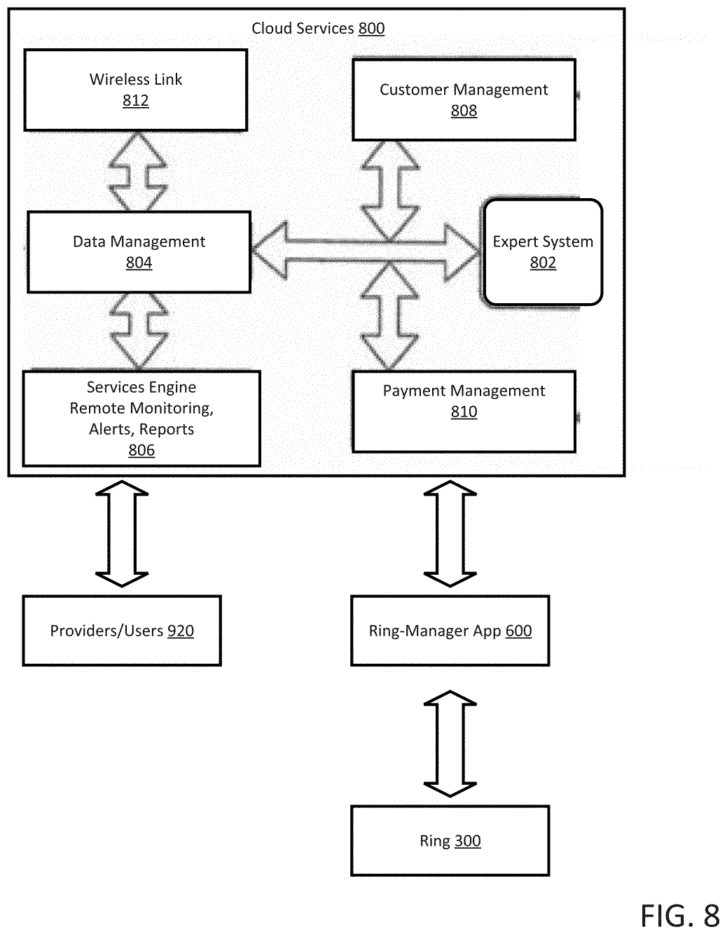

Cloud services 800, represented in FIG. 8, includes an expert system 802, data management 804, a services engine 806, customer management 808, payment management 810, and a wireless link 812. Data management 804 provides secure access to and maintenance of sensed data and results derived therefrom, as well as customer profile data and health data received from providers. Services engine 806 provides for remote monitoring, alerts, and reports based on the analysis provided by expert system 802. Customer management 808 provides for new customer accounts and for managing and updating existing customer accounts. Payment management 810 provides for financial accounting and payments. Wireless or wired link 812 provides for communication with ring-manager apps and with providers and other users.

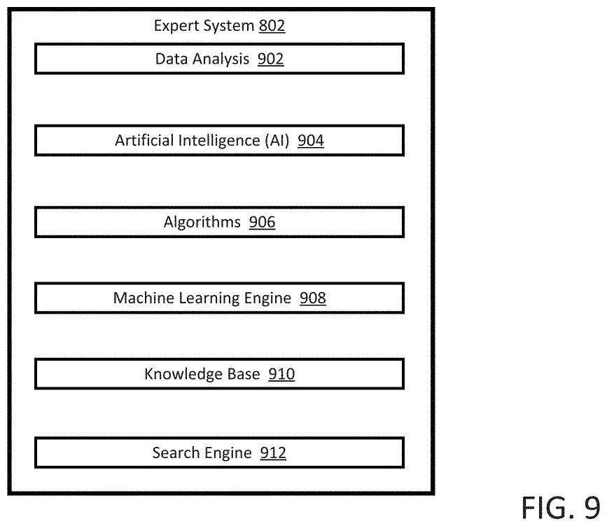

Expert system 802, as shown in FIG. 9 includes a data analysis module 902, an artificial intelligence module 904, algorithms 906, a machine-learning engine 908, a knowledge base 910, and a search engine 912. Data analysis module 904 analyzes incoming app-processed sensor data in relation to previously collected data from the respective ring-wearer and from normative data, some of which may be obtained from other ring-wearers and some of which may be obtained by references in knowledge base 912 or obtained using search engine 912. To assist in data analysis, module 902 can leverage artificial intelligence 904 and algorithms 906. Machine-learning engine is used to update algorithms 906, e.g., based on feedback from providers.

Herein, all art labeled “prior art”, if any, is admitted prior art; art not labelled “prior art”, if any, is not admitted prior art. The illustrated embodiments, variations thereupon, and modifications thereto are provided for by the present invention, the scope of which is defined by the following claims.

What is claimed is:

1. A cloud-connected ring-based sensor process comprising:
mounting ring on finger of a ring-wearer;
collecting first sensor data using a sensor included in the ring while it is worn on the finger of the ring-wearer;
transferring second sensor data from ring to a ring-manager application running on a mobile device, the second sensor data being the same as or being based on the first sensor data;
transferring third sensor data from the ring-manager application to cloud services, the third sensor data being the same as or being based on the second sensor data;
transferring fourth sensor data from the cloud services to a provider, the fourth sensor data being the same as or being based on the third sensor data;
transferring first provider information from the provider to the cloud services;
transferring second provider information from cloud services to the ring-manager application, the second provider information being the same as or being based on the first provider information;
displaying, by the ring-manager application, third provider information to the ring-wearer, the third provider information being the same as or being based on the second provider information.

2. The cloud-connected ring-based sensor process of claim 1 wherein the first sensor data includes heart-health data regarding the ring-wearer.

3. The cloud-connected ring-based sensor process of claim 1 further comprising deriving inter-beat interval (IBI) data regarding time intervals between heartbeats of the ring-wearer, the IBI data being based on the first sensor data.

4. The cloud-connected ring-based sensor process of claim 1 further comprising determining a number of missed heartbeats based on the IBI data.

5. The cloud-connected ring-based sensor process of claim 1 wherein the second sensor data indicates a heart rate of the ring-wearer.

6. The cloud-connected ring-based sensor process of claim 1 wherein the second sensor data indicates a heart-rate variability of the ring-wearer.

7. The cloud-connected ring-based sensor process of claim 1 wherein the second sensor data indicates a blood oxygen level of the ring-wearer.

8. The cloud-connected ring-based sensor process of claim 1 wherein the second sensor data indicates a body temperature of the ring-wearer.

9. The cloud-connected ring-based sensor process of claim 1 wherein the fourth sensor data indicates a trend in heart-rate variability of the ring-wearer.

10. The cloud-connected ring-based sensor process of claim 1 wherein the third provider information includes medical advice to the ring-wearer.

11. A cloud-connected ring-based sensor system comprising:
a ring including a sensor and a wireless interface, the sensor providing for collecting first sensor data, the wireless interface providing for transferring second sensor data to a ring-manager application, the second sensor data being the same as or based on the first sensor data;
cloud services for transmitting fourth sensor data to a provider, receiving first provider information from the provider, and transferring second provider information to the ring-manager application, the second provider information being the same as or being based on the first provider information;
the ring-manager application, which, when executed by a processor, transfers third sensor data to the cloud services and displays third provider information to the ring-wearer, the third sensor data being the same as or being based on the second sensor data, the fourth sensor data being the same as or being based on the third sensor data, the third provider information being the same as or being based on the second provider information.

12. The cloud-connected ring-based sensor system of claim 11 wherein the first sensor data includes heart-health data regarding the ring-wearer.

13. The cloud-connected ring-based sensor system of claim 11 wherein the ring includes a processor for deriving, from the first sensor data, inter-beat interval (IBI) data regarding time intervals between heartbeats of the ring-wearer.

14. The cloud-connected ring-based sensor system of claim 11 wherein the second sensor data indicates a number of missed heartbeats.

15. The cloud-connected ring-based sensor system of claim B1 wherein the second sensor data indicates a heart rate of the ring-wearer.

16. The cloud-connected ring-based sensor system of claim 11 wherein the second sensor data indicates a heart-rate variability of the ring-wearer.

17. The cloud-connected ring-based sensor system of claim 11 wherein the third provider information includes medical advice to the ring-wearer.

18. A heart-health sensor system comprising a ring including:
a heartbeat sensor for probing for and detecting heartbeats of a ring-wearer when the ring is mounted on a finger of the ring-wearer;
a processor for generating, based on a set of one or more algorithms, heart-health data based on heartbeat detections;
memory for storing the heart-health data and the algorithms;
a wireless interface for transmitting the heart-health data;
a power source for powering the heartbeat sensor, the processor, the memory, and the wireless interface; and
a power manager having an active mode and a standby mode, the power manager, while in its active mode, causing the heartbeat sensor to probe for heartbeats more than once per second, the power manager, while in its active mode, causing the heartbeat sensor to probe for heartbeats at an average rate of less than once per second.

19. The heart-health sensor system of claim 18 wherein, the power manager, in the event that a enable criterion is met while the power manager is in its standby mode, switches to its active mode, the enable criterion including detection of a heartbeat.

20. The heart-health sensor system of claim 18 wherein, the power manager, in the event that a disable criterion is met while the power manager is in its active mode, switches to its standby mode, the disable criterion including a failure to detect a heartbeat for a period of time.
